(19) United States
(12) Patent Application Publication
(10) Pub. No.: US 2025/0229003 A1
(43) Pub. Date: Jul. 17, 2025

(54) DISSOLVABLE CALCIUM ALGINATE MICROFIBERS PRODUCED VIA IMMERSED MICROFLUIDIC SPINNING TOWARDS FABRICATION OF EMBEDDED MICROFLUIDIC NETWORKS

(71) Applicant: THE REGENTS OF THE UNIVERSITY OF CALIFORNIA, Oakland, CA (US)

(72) Inventors: Lawrence Kulinsky, Irvine, CA (US); Tuo Zhou, Irvine, CA (US); Sahar NajafiKhoshnoo, Irvine, CA (US); Rahim Esfandyarpour, Irvine, CA (US)

(21) Appl. No.: 19/020,045

(22) Filed: Jan. 14, 2025

Related U.S. Application Data
(60) Provisional application No. 63/621,730, filed on Jan. 17, 2024.

(51) Int. Cl. A61L 27/50; A61L 27/20; A61L 27/38; C08L 5/04

(52) U.S. Cl. CPC A61L 27/507; A61L 27/20; A61L 27/38; C08L 5/04

(57) ABSTRACT

Methods for generating a vascularized tissue engineering construct that involves using the immersed microfluidic spinning to create a network of calcium alginate fibers. These fibers are covered by a layer of the cell-laden hydrogel, another layer of fibers is spun upon the layer of hydrogel, and another layer of hydrogel is deposited over that layer of calcium alginate fibers. DMEM/EDTA/DSC solution is used to dissolve the calcium alginate fibers that would then leave a channel of several hundred microns in diameter inside the cell-laden hydrogel. This microfluidic network can be used to flow the nutrients to the cells and waste products away from the cells inside the hydrogel construct, assisting in the creation of the vascularized tissue construct.

### CROSS-REFERENCE TO RELATED APPLICATIONS

This application is a non-provisional and claims benefit of U.S. Provisional Application No. 63/621,730 filed Jan. 17, 2024, the specification(s) of which is/are incorporated herein in their entirety by reference.

### FIELD OF THE INVENTION

The present invention features a novel manufacturing process for creating an embedded microfluidic network inside cell-laden hydrogel tissue engineering constructs where this fluidic network can be used for delivery of nutrients to the cells and removal of cell activity waste, allowing for cells to survive while the slow-growing vascularized network is built.

### BACKGROUND OF THE INVENTION

Micro- and nanofibers characterized by high surface area, predictable geometry, long aspect ratio, and high speed of production have important applications in a wide range of fields from composite materials and wearable electronics to water collection and tissue engineering. In tissue engineering, one of the critical problems is that of tissue vascularization. Vascularization is the creation of a network of fluidic channels of high density for delivery of nutrients to the cells of the bioengineered tissue, as well as waste extraction from the cells, in the period from days to weeks (depending on the size of the implanted tissue) before the body's own blood vessels are developing within a tissue construct. If the cells do not have access to the fluidic network, then they will die within a couple of weeks, leading to tissue necrosis.

One of the approaches for the fabricated microfluidic network within a tissue construct can be the creation of the sacrificial network of microfibers inside the cell-laden gel matrix. In cases when the fiber network is made from natural carbohydrate alginate that can be dissolved in ethylenediaminetetraacetic acid (EDTA), the dissolution of the alginate fibers would leave behind hollow microfluidic channels suitable for transport of nutrients to the cells of the tissue.

Previous methods such as template and 3D printing are not ideal for creating microfluidic networks because 1) they are slow processes, and 2) the dense mesh of microfibers would make it difficult for the cells to access the inside. Hence, the challenge lies in fabricating a microfluidic network quickly and in three dimensions.

Out of two main methods for the fabrication of micro and nanochannels-electrospinning and fluidic spinning, only the fluidic spinning is capable of producing fibers with diameters larger than several microns. In order to utilize fast transport of nutrients to the cells and to enable fast fiber dissolution, fibers with diameters in the range between several microns to hundreds of microns are necessary. Therefore, the present invention focused on using fluidic spinning for the production of alginate microfibers.

Recent review surveys various manufacturing technologies for the production of calcium alginate fibers including freeze-drying, wet-spinning technology, immersive centrifugal jet spinning technique, and microfluidic spinning technology. Freeze-drying is, by itself, not a method of producing the fibers, but rather a post-fabrication technique to introduce porosity within alginate fibers during the freeze-drying stage once fibers are produced.

The fluidic spinning of microfibers involves the flow of liquid solution in a microchannel and that solution is solidified upon either exposure to chemical crosslinkers or via UV light acting on a photoinitiator within the solution (i.e., via photopolymerization). Typically, photopolymerized material is more difficult to dissolve and thus, the photopolymerization technique is less suitable for the fabrication of sacrificial microfiber networks. As such, the present invention has focused on studying chemically cross-linkable systems of alginate that are solidified upon exposure to CaCl₂) solution to produce calcium alginate microfibers.

In wet spinning technology, the alginate fibers ejected via a spinneret are drawn through the chemical cross-linking solution. The immersive centrifugal/rotary jet spinning technique is similar to wet spinning, but the resulting fibers are collected on a rotary drum or deposited along the walls of a funnel. The most popular approaches to controlled microfluidic production of calcium alginate microfibers are using coaxial needles (where the central core carries an alginate solution, and the sheath flow is that of CaCl₂) cross-linking solution) or similarly structured microfluidic chips.

In the calcium alginate microfiber production approaches discussed so far, such as wet-spinning technology, immersive centrifugal rotary jet technology, and microfluidic spinning technology, the goal is to produce aligned fibers (such as in immersive centrifugal spinning and wet-spinning) or to deposit fibers in pre-designed pattern (via microfluidic spinning). The present invention proposes a variation of the traditional wet-spinning approach where the alginate fiber is not drawn through the cross-linking solution to obtain straight fibers or rolling the resulting fibers onto a drum.

### BRIEF SUMMARY OF THE INVENTION

In some aspects, the present invention features immersed microfluidic spinning of the calcium alginate microfibers that is instrumental for design, fabrication, and optimization of the sacrificial microfiber network towards production of the advanced vascularized tissue engineered constructs.

In some embodiments, the present invention features a vascularized tissue construct produced by an immersed microfluidic spinning process. This process may involve injecting a microfiber material through a needle that is immersed in a cross-linking solution such that as the microfiber material is ejected from the needle and exposed to the crosslinking solution, it becomes solidified to form a fiber network. The fabrication process may further include removing the fiber network from the crosslinking solution, embedding the fiber network into a cell-laden gel matrix, polymerizing the cell-laden gel matrix, and dissolving said fiber network to leave behind microfluidic channels in the cell-laden gel matrix suitable for vascularization. The resulting vascularization tissue construct may comprise microfluidic channels suitable for delivering nutrients to the cells and transporting waste away from the cells.

In the proposed immersed microfluidic spinning technology, the alginate fibers are injected through the needle into the cross-linking solution where the fiber can be either deposited in relatively straight segments or to produce a densely coiled stochastic fiber mesh. Parameters that determine the topology of the resulting calcium alginate fiber network are discussed herein.

In some embodiments, the method uses a single core needle attached to a syringe filled with an alginate solution. The syringe's volumetric displacement is controlled by a pump. The needle of the syringe is immersed in a bath of CaCl₂ solution. As the liquid jet of alginate is ejected from the needle, it is exposed to the cross-linking solution and is getting solidified. The influence of the needle size, the volumetric flow rate of the alginate solution, and the weight % concentration of the alginate solution on the diameter of the resulting calcium alginate microfiber are further described herein.

Other techniques for producing alginate fibers, such as wet spinning or on-chip microfluidic spinning, do not produce a stochastic network of fibers that can be dissolved to leave behind hollow microfluidic networks of channels appropriate for applications such as vascularized tissue constructs.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a process for producing and transporting a fiber network with the immersed microfluidic spinning of the present invention. The meshed cage (a) is inserted inside the beaker with the calcium alginate cross-linking solution (b), followed by the immersed microfluidic spinning of the fiber inside the meshed cage (c), and removal of the cage with the produced fiber network (d) that is now ready for transport to a separate location.

FIG. 1B shows the immersed microfluidic spinning setup that includes a pump displacing the plunger of a syringe filled with a sodium alginate solution. The needle of the syringe is immersed in a bath of calcium chloride.

FIG. 2 shows the images of fibers generated by the immersed microfluidic spinning.

FIG. 3 shows the dependence of the fiber diameter on the gauge of the needles for various concentrations of sodium alginate with the sodium alginate flow rate kept at 1 ml/min. The error bars represent the standard deviation of the measured diameters.

FIG. 4 shows the dependence of the fiber diameter on the flow rate of the 2 wt % sodium alginate. There is no pronounced trend for the resulting fiber diameter with an increase of the flow rate from 1 ml/min to 2.7 ml/min. The error bars represent the standard deviation of the measurements.

FIG. 5 shows the dependence of the fiber diameter on the concentration of the sodium alginate with the flow rate of 1 ml/min. The error bars represent the standard deviation of the measurements.

FIG. 6 shows the observed topology of the fiber deposited via immersed microfluidic spinning. In both cases, 1 ml/min volumetric flow rate of the sodium alginate was used. The left picture represents the spinning of 6 wt % sodium alginate with a 20 gage needle and the right picture presents the spinning of 4 wt % sodium alginate with a 25 gauge needle.

FIG. 7 shows the dissolution of 8% alginate fibers produced with the immersed microfluidic spinning. The top row of images is a timed sequence of the dissolution of 240-micron diameter fiber, the middle row contains images of the dissolution of 680-micron fiber, while the bottom row contains images of 800-micron diameter fibers. All fibers had a length of 1 mm and were covered by a layer of hydrogel. The DMEM/EDTA/DSC solution was used to dissolve the calcium alginate fibers. The dissolution was observed in the microscope and a timed sequence of images was taken. The initial images before dissolution are on the left, the middle images correspond approximately to 10 minutes after dissolution started. The leftmost images are the images that did not change with time (i.e., the fiber dissolution was completed)—for 240-micron diameter fiber it took 20 minutes to dissolve, while for 680- and 800-micron diameter fibers it took around 30 minutes to dissolve.

### DETAILED DESCRIPTION OF THE INVENTION

Fabrication of micro- and nanofibers are critical for a wide range of applications from microelectronics to biotechnology. Alginate microfibers with diameters of tens to hundreds of microns play an important role in tissue engineering and fibers of these diameters are impossible to fabricate via electrospinning and can only be produced via fluidic spinning. Typically, microfluidic spinning based on photopolymerization produces fibers that are not easily dissolvable, while fluidic spinning with chemical cross-linking employs complex setups of microfabricated chips or coaxial needles, aimed at precise control of the fiber diameter, but adds a significant cost and complexity to the microfluidic setup.

Referring to the figures, in some embodiments, the present invention features a method of fabricating a microfluidic network. The method may comprise injecting a microfiber material through a needle and into a cross-linking solution so that the microfiber material becomes solidified as it is injected into the cross-linking solution. In some embodiments, the microfiber material can be deposited in relatively straight segments or in loops to produce a densely coiled stochastic fiber network.

According to other embodiments, the present invention features a method of fabricating a tissue construct. The method may comprise injecting a microfiber material through a needle and into a cross-linking solution such that as the microfiber material is ejected from the needle and exposed to the crosslinking solution, it becomes solidified to form a fiber network. The method further comprises removing the fiber network from the crosslinking solution, embedding the fiber network into a cell-laden hydrogel, polymerizing the cell-laden hydrogel, and dissolving said fiber network to leave behind microfluidic channels in the cell-laden hydrogel suitable for vascularization. In other preferred embodiments, the microfluidic channels are suitable for delivering nutrients to the cells and transporting waste away from the cells.

In some embodiments, the needle may be immersed in the cross-linking solution. In other embodiments, the needle may be positioned above the cross-linking solution. For example, the needle may be hovering at or near the top surface of the cross-linking solution. In non-limiting embodiments, the microfiber material may be deposited in relatively straight segments or loops to produce a densely coiled stochastic fiber.

In some embodiments, the diameter of the microfiber material segments or loops is smaller than an internal diameter of the needle. In a non-limiting embodiment, a diameter of the microfiber material segments or loops can range from about 10 microns to about 100 microns. In another non-limiting embodiment, the diameter of the microfiber material segments or loops can range from about 100 microns to about 500 microns, or about 300 microns to about 800 microns, or about 500 microns to about 1 mm.

In other embodiments, the microfiber material may comprise a carbohydrate alginate or a calcium alginate. In some other embodiments, the microfiber material may comprise agar-agar, sodium alginate or other soluble materials. In some embodiments, the fiber network can be dissolved in ethylenediaminetetraacetic acid (EDTA). Examples of other materials for tissue constructs may include, but is not limited to, synthetic polymers, gelatin, etc.

In a non-limiting embodiment, the cross-linking solution may be a calcium chloride solution. Other cross-linking solutions can be used that contain divalent cations such as Ba (2+), Sr (2+), etc. For example, the cross-linking solution may be a solution containing divalent cations and the microfiber material may be sodium alginate.

In other embodiments, the cells that can be used in accordance with the present invention include, but are not limited to, a wide variety of biological cells such as liver cells, kidney cells, etc.

In some embodiments, the cell-laden hydrogel may be polymerized by photopolymerization. In other embodiments, the hydrogel can include photoinitiators that aid in the photopolymerization. In some other embodiments, an ultraviolet light may be used to photopolymerize the cell-laden hydrogel.

According to some embodiments, the present invention features a vascularized tissue construct produced by a method comprising injecting a microfiber material through a needle that is immersed in a cross-linking solution such that as the microfiber material is ejected from the needle and exposed to the crosslinking solution, it becomes solidified to form a fiber network. The fabrication process may further include removing the fiber network from the crosslinking solution, embedding the fiber network into a cell-laden gel matrix, polymerizing the cell-laden gel matrix, and dissolving said fiber network to leave behind microfluidic channels in the cell-laden gel matrix suitable for vascularization. The resulting vascularization tissue construct may comprise microfluidic channels suitable for delivering nutrients to the cells and transporting waste away from the cells.

Without wishing to be limited to a particular example, theory, or mechanism, the present invention demonstrated the immersed microfluidic spinning where a calcium alginate microfiber is produced via displacement of alginate solution through a single needle that is immersed in a cross-linking bath of calcium chloride solution. The resulting diameter of the fiber is characterized, and fiber diameter and topology of the deposited fiber are related to the concentration of the alginate solution (2, 4, and 6 wt %), needle gauge (30g, 25g, and 20g), and the volumetric flow rate of the alginate solution (1 ml/min, 2 ml/min, and 2.7 ml/min). The resulting fiber diameter is smaller than the internal diameter of the needle and this dependence is explained by the continuity of the flow and increased rate of fall of the liquid jet upon its issuing from the needle. Without wishing to limit the present invention, the fiber diameter (demonstrated diameter of fibers ranges from 100 microns to 1 mm) depends weakly on the volumetric flow rate and depends strongly on the needle diameter. In other embodiments, it also seems that for smaller needle size, a greater concentration of alginate results in smaller diameter fibers and that this trend is not evident as needle diameter is increased.

In terms of the topology of the deposited fiber, the higher wt % alginate fiber produces larger loops, while smaller wt % alginate solution yields a denser topology of the overlaid fiber loops. These fibers can be dissolved in DMEM/EDTA/DSC solution in 20-30 minutes (depending on the fiber diameter), leaving behind the hollow channels in the hydrogel matrix. The demonstrated setup of the immersed microfluidic spinning of the calcium alginate microfibers can be useful for creating tissue constructs, including vascularized tissue implants.

### Example

The following is a non-limiting example of the present invention. It is to be understood that said example is not intended to limit the present invention in any way. Equivalents or substitutes are within the scope of the present invention.

### Materials and Methods

Solutions of sodium alginate of three concentrations (2 wt %, 4 wt %, 6 wt %) were prepared by dissolving alginic acid sodium salt (Sigma-Aldrich, MO, USA) in Deionized (DI) water followed by stirring for 3 hours at 60° C. A 1 wt % calcium chloride solution was prepared by dissolving the calcium chloride powder (Sigma-Aldrich, MO, USA) into the DI water, followed by vortex mixing for 2 mins.

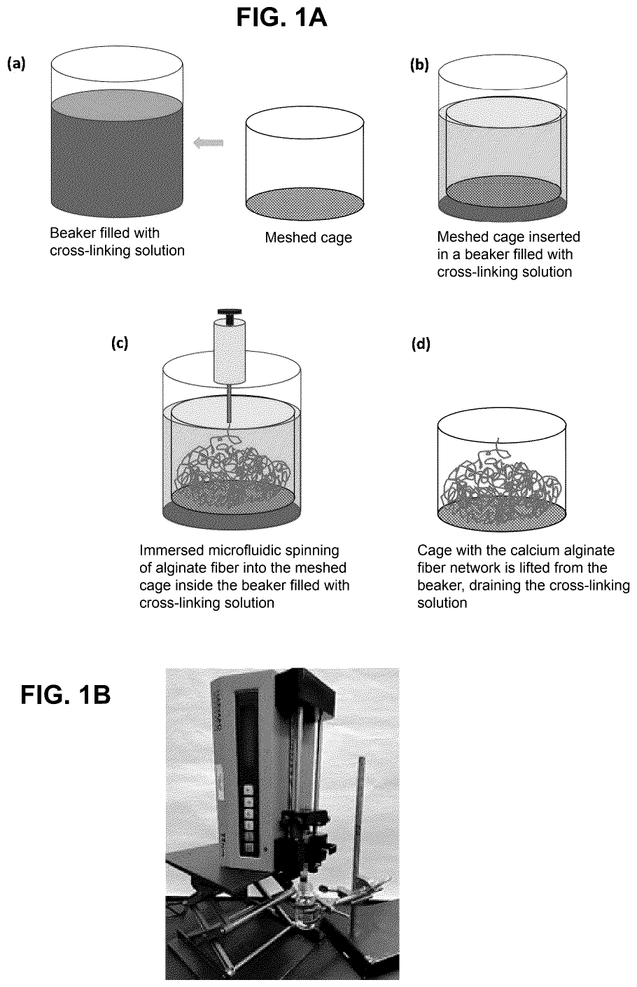

FIG. 1B demonstrates the setup of the immersed microfluidic spinning. In this setup, a syringe pump (Harvard Apparatus, MA, USA) is fitted with a 3 ml syringe (BD, NJ, USA) with an attached single-core needle (Jensen Global, CA, USA). Three different sets of needles with inner diameters of 30g (810 microns), 25g (455 microns), and 20g (250 microns) were used to generate fibers. Three different volumetric flow rates of 1 ml/min, 2 ml/min, and 2.7 ml/min (maximum achievable by the pump) were utilized to study fabrication of the spun microfibers. The sodium alginate solution was ejected out of the needle that was immersed in a vial of 1 wt % calcium chloride cross-linking solution.

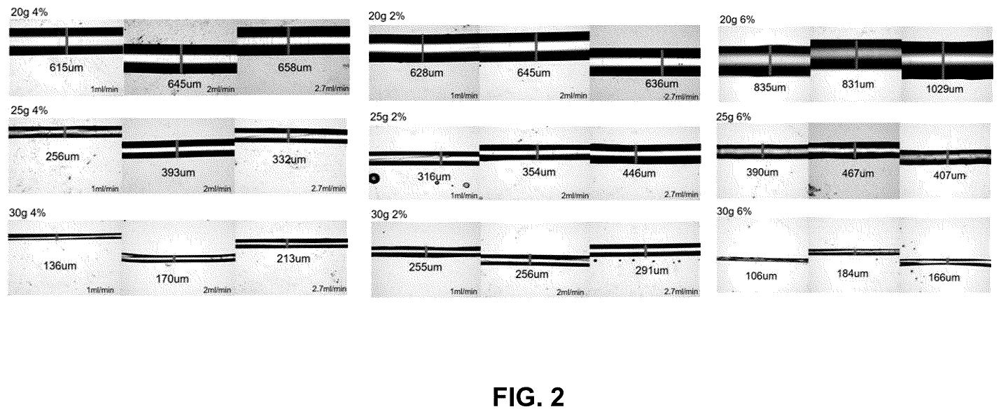

The cross-linked fibers were collected from the vial with tweezers and studied under the high-power Nikon Eclipse microscope (Nikon, Japan). The microfabricated gauge with 100-micron gap was imaged next to the fibers (not pictured in FIG. 2) to verify the fidelity of the measurements. The SPOT Basic Imaging software (Spot Imaging, MI, USA) was used to observe and measure the diameter of the fibers.

In order to study the dissolution rate of the fibers, 0.2 g of Ethylenediaminetetraacetic acid (EDTA, Thermo Fisher Scientific, Waltham, MA, USA) and 0.3 g of Disodium citrate (DSC, Sigma-Aldrich, St. Louis, MO, USA) were added to the solution of 50 ml of Dulbecco's Modified Eagle Medium (DMEM, Thermo Fisher Scientific, Waltham, MA, USA). The fibers were prepared by the immersed fluidic spinning technique described above. The spun fibers were then extracted by tweezers from the calcium chloride cross-linking solution. Then, a 1 mm long piece of the fiber was covered by the hydrogel. In order to avoid studying the parts of the fiber distorted by the handling, the ends of the longer piece of fiber were cut off from the ends where the tweezers compressed the fibers and we studied the length of the fiber not affected by the handling process.

The Gelatin methacryloyl (GelMA) hydrogel was synthesized by dissolving type A gelatin from porcine skin (Sigma-Aldrich, St. Louis, MO, USA) at 10% (w/v) in Dulbecco's phosphate-buffered saline (DPBS) (Thermo Fisher Scientific, Waltham, MA, USA) and stirring at 50° C. for one hour. Methacrylic acid (Sigma-Aldrich, St. Louis, MO, USA) was added to the solution at 10% (v/v) and allowed to react for one hour. The reaction was finished by adding 5× volumes of DPBS. The resulting solution was dialyzed for seven days with distilled water at 37° C. to remove unreacted methacrylic anhydride using dialysis membranes, then changing the water twice daily. The resulting solution was frozen and lyophilized for five days and stored at −80° C. for future use. Lyophilized GelMA was dissolved at 5% (w/v) in DPBS at 70° C. in a water bath containing 0.2% (w/v) lithium phenyl-2, 4, 6-trimethylbenzoylphosphinate (LAP) (Allevi, Inc., Philadelphia, PA, USA) as the photoinitiator.

The fiber was embedded in the hydrogel, and then exposed to UV light (405 nm) to crosslink the GelMA hydrogel. The hydrogel sample with embedded fiber was then covered by the prepared DMEM/EDTA/DSC solution for fiber dissolution. Visual observation of the fiber under the microscope was performed periodically to record when the fiber was completely dissolved, and the hollow channel was left in the hydrogel.

### Results and Discussion

### Dependence of the Fiber Diameter on Sodium Alginate Flow Rate, Alginate Wt %, and Needle Gauge

FIG. 2 shows optical pictures of the produced fibers while Table 1 summarizes the average fiber diameters for each set of parameters. The images of the fibers are grouped into the sets of pictures of fibers made from sodium alginate of 2, 4, and 6 wt % where each row of images presents the results of spinning with a different needle gauge (of 20g, 25g, and 30g, respectively). In each set of three sequential pictures, these images represent the increase in the volumetric flow rate from 1 ml/min to 2 ml/min to 2.7 ml/min. The white regions on the images of most of the fibers do not mean that these are hollow fibers but are indicative of the fact that for larger fibers, top sections of the fiber are not in focus.

TABLE 1
Mean values and standard deviations of diameters of the
cross-linked calcium alginate fibers generated via immersed
microfluidic spinning using different volumetric flow rates
of sodium alginate, different concentrations of sodium
alginate solution, and the gauges of the needles.

| 1 ml/min | | 2 ml/min | | 2.7 ml/min | |
|---|---|---|---|---|---|
| Needle | Diameter of | Needle | Diameter of | Needle | Diameter of |
| size | fiber ( [symbol] m) | size | fiber ( [symbol] m) | size | fiber ( [symbol] m) |

2% alginate

| | | | | | |
|---|---|---|---|---|---|
| 30 g | 255 ± 4 | 30 g | 256 ± 20 | 30 g | 291 ± 13 |
| 25 g | 316 ± 13 | 25 g | 354 ± 13 | 25 g | 446 ± 8 |
| 20 g | 628 ± 8 | 20 g | 645 ± 13 | 20 g | 636 ± 8 |

4% alginate

| | | | | | |
|---|---|---|---|---|---|
| 30 g | 136 ± 19 | 30 g | 170 ± 30 | 30 g | 213 ± 66 |
| 25 g | 256 ± 2 | 25 g | 393 ± 15 | 25 g | 332 ± 2 |
| 20 g | 615 ± 22 | 20 g | 645 ± 7 | 20 g | 658 ± 8 |

6% alginate

| | | | | | |
|---|---|---|---|---|---|
| 30 g | 106 ± 3 | 30 g | 184 ± 9 | 30 g | 166 ± 3 |
| 25 g | 390 ± 12 | 25 g | 467 ± 8 | 25 g | 407 ± 8 |
| 20 g | 835 ± 26 | 20 g | 831 ± 17 | 20 g | 1029 ± 15 |

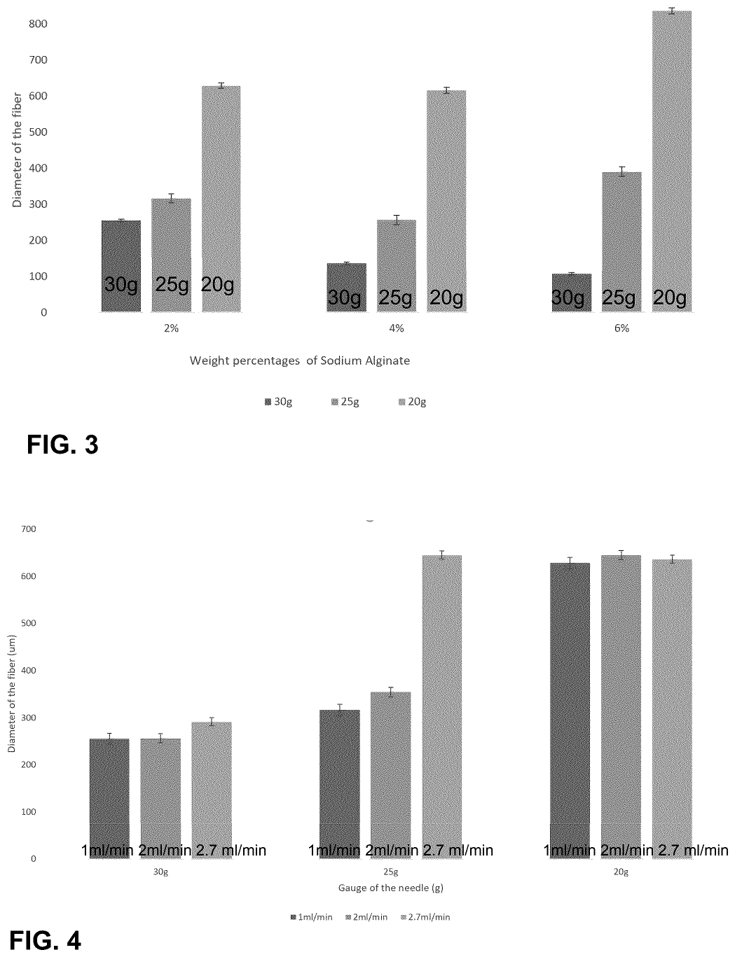

It is clear from the observed data that the gauge of the needle plays a role in the resulting diameter of the spun microfiber—the smaller the inner diameter of the needle (the greater its gauge)—the smaller the resulting fiber diameter. FIG. 3 illustrates the dependence of the fiber diameter on the gauge of the needle. In order to demonstrate the trend, a specific volumetric flow rate of the calcium alginate of 1 ml/min was selected. It was observed that for all concentrations of sodium alginate, the use of the smaller diameter needles (larger gauge needles) results in smaller diameter calcium alginate fibers. The fiber diameters given in FIGS. 3, 4, and 5, as well as in Table 1 are the mean diameters measured using three sets of data and the error bars represent the measured standard deviation.

It is important to observe that the fiber diameters are smaller than the inner diameters of the needles. The flow rate of the sodium alginate does not noticeably affect the resulting fiber diameter as evidenced by the plot presented in FIG. 4.

It was observed that there was a significant decrease in fiber diameter produced with a 30g needle as the sodium alginate concentration was increased from 2 wt % to 6 wt %. For example, for the given flow rate of 1 ml/min the fiber diameter is reduced from about 250 microns to about 100 microns (see FIG. 5). This is a promising trend that should be studied more closely given the fact that the increase in the concentration of the sodium alginate solution does not always result in smaller diameter fibers when the larger inner diameter needles (20g, 25g) are used.

### Topology of the Deposited Microfluidically Spun Fiber Network

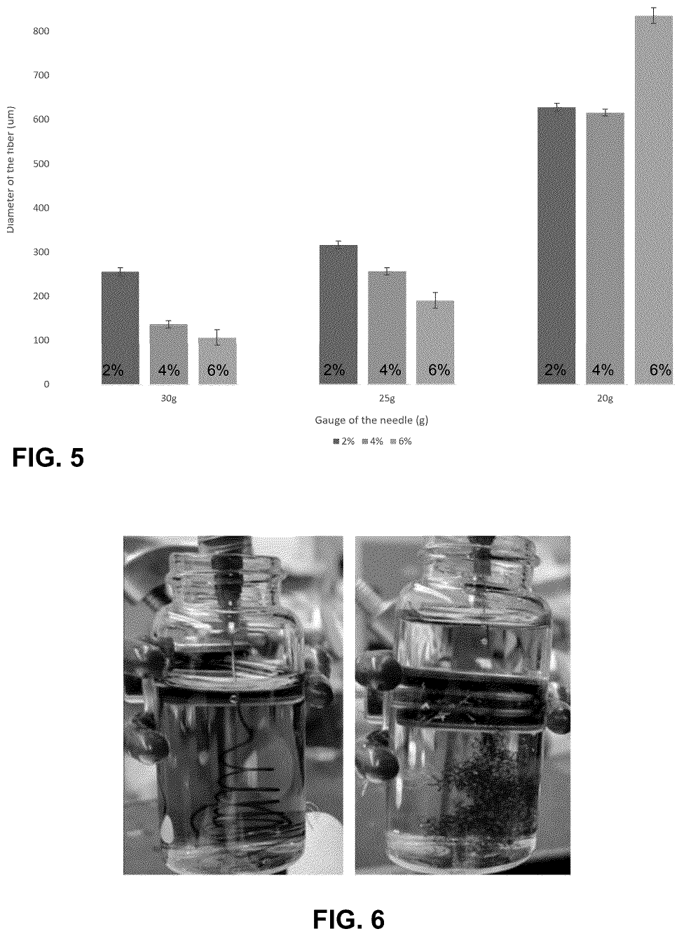

For many applications, including tissue engineering, the topology of the deposited fiber could play an important role. It was observed (see FIG. 6) that for the same flow rate (1 ml/min), higher concentration (6 wt %) of sodium alginate produces larger loops of the deposited fiber, while the smaller (4 wt %) concentration of sodium alginate produces tighter loops of fiber, more appropriate for higher density of the deposited fibers. The blue food dye was added to the sodium alginate solution to facilitate the observation of the deposited fiber networks.

In some embodiments, the topology of the deposited networks can be optimized (based on the application) by varying the concentration of the alginate solution and other parameters. Without wishing to limit the present invention, it is believed that a higher concentration of the alginate solution results in a denser fiber, imparting it higher inertia, explaining the observed trajectory of the resulting larger fiber coils.

Once the fiber network is produced, the cross-linking solution can be drained or aspirated, and cell-laden hydrogel can be added to the fiber network. If the whole fiber network is desired to be transferred to a separate location once it is created, then the deposition can be conducted within the plastic or metallic mesh inserted inside the glass beaker. Thus, after the generation of the fiber network, that mesh can be lifted from the glass beaker, draining the cross-linking solution, and transferring that fiber network to a different volume (see FIG. 1A).

It was expected that due to the dynamic nature of the deposition of the fiber network and stretching of the fibers as they fall into the cross-linking solution, there will be some variation in the fiber diameter (see Table 1 for the standard deviations of the diameter of the produced fibers). Therefore, if precise control of the fiber diameter is desired, then such spinning techniques as wet spinning or microfluidic spinning surveyed in the Introduction section above will be a more appropriate fabrication approach.

Deposited Fiber Diameter is Smaller than the Inner Diameter of the Needle

The narrowing of the fluid jet issuing from the needle into another fluid can be observed in many applications, including in water flowing from the kitchen faucet. The observed phenomenon relates to the acceleration on the falling jet after it issues from the constraining pipe. As the continuity of the jet is preserved and the same volume of the jet now goes through a larger distance under the gravitational acceleration, the narrowing of the jet is observed.

The combination of the continuity equation and the Navier-Stokes equation in the falling jet constrained by the surface tension can be described by the modified Bernoulli equation in the form of Eqn. 1:

$$P + \frac{α}{2} ρ {v}^{2} + ρ gz + γ ( \frac{\partial A}{\partial V} ) + {ω}_{los} = const . \qquad ( 1 )$$

where P is the applied pressure and u is the mean velocity of the falling jet, g is the gravitational acceleration, z is the distance from the tip of the needle, [symbol] is the density of the falling jet, © is the coefficient of the surface tension, [symbol] ₗₒₛ is the dissipation energy due to viscous resistance, A is the cross-sectional area of the jet at a specific coordinate z, dV is the infinitesimal volume of the fluid element, and [symbol] is the coefficient describing the velocity profile distribution within the jet with alpha of 1 representing the uniform velocity and alpha of 2 representing the parabolic velocity profile.

This analysis was successfully performed for the falling water jet in the air. The analogous analysis might not yield satisfactory results due to the presence of the fluid significantly more viscous than air that consequently will absorb a significant portion of the energy of the falling jet. However, the main underlying physical principles remain valid, and they explain the narrowing of the jet issuing from the needle during the immersed microfluidic spinning.

### Dissolution of the Fluidically Spun Microfibers

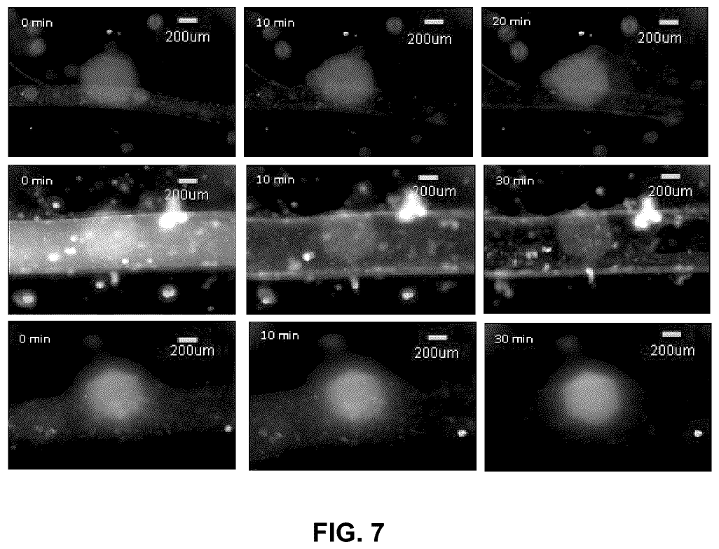

The segments of 1 mm fibers of three diameters—240 [symbol] m, 680 [symbol] m, and 800 [symbol] m spun from 8% alginate (see the Materials and Methods section above) were covered by hydrogel followed by immersion into the DMEM/EDTA/DSC solution as described in the Materials and Methods. It is difficult to observe the fiber dissolution process since both the calcium alginate fiber and the hydrogel matrix into which the fiber is embedded are both soft transparent media. The inventors discovered that observation of nano inclusions and/or tiny bubbles of the air trapped inside the alginate fibers is the most convenient way to discover the process of fiber dissolution. When the fiber dissolves, these inclusions can move and are washed away. The process of elimination of the inclusions as the fiber is dissolved can be followed by observing the timed sequence of images of the fiber segments presented in FIG. 7.

All fibers had a length of 1 mm and were covered by a layer of hydrogel. The DMEM/EDTA/DSC solution was used to dissolve the calcium alginate fibers. The dissolution was observed in the microscope and a timed sequence of images was taken. Images before the dissolution of fibers are presented in the left column of images, while the images in the middle column were taken after 10 minutes of immersing the fibers in the dissolving solution. The leftmost images are the images that did not change with time (i.e., the fiber dissolution was completed). The images in the rightmost column were taken after 20 minutes for 240-micron fibers and after 30 minutes for 680-micron and 800-micron fibers when there no longer was a change in the appearance of the fiber and we could conclude that the dissolution process was completed. Therefore, it was concluded that the 800- and 680-micron fibers were dissolved in about 30 minutes, while the 240-micron diameter fiber segment was dissolved in about 20 minutes.

### CONCLUSIONS

The present invention provides a method of immersed microfluidic spinning capable of generating calcium alginate fibers with diameters between 100 microns and 1000 microns. A series of experiments were performed where the influence of three parameters—the volumetric flow rate of sodium alginate (1 ml/min, 2 ml/min, and 2.7 ml/min), the concentration of sodium alginate (2 wt %, 4 wt %, and 6 wt %), and the inner diameter of the needle (20g, 25g, and 30g) were studied and their influence on the resulting calcium alginate fiber diameter was reported. The study has demonstrated that the flow rate of the alginate does not significantly affect the resulting diameter of the fibers, that the reduction in the size of the needle decreased the resulting diameter of the fibers, and that an increase in the sodium alginate concentration decreases fiber diameter for 30g needle, but there is no pronounced effect for larger needles. The topology of the deposited calcium alginate fiber network depends on the concentration of the sodium alginate, with tighter, denser coils of fibers being deposited when the smaller wt % of sodium alginate was used. These fibers can be dissolved in DMEM/EDTA/DSC solution in 20 to 30 minutes (depending on the fiber diameter), leaving behind the hollow channels in the hydrogel matrix.

The immersed microfluidic spinning technology generating dissolvable calcium alginate microfibers with a diameter of several hundred microns that can be deposited in tight small coiled stochastic networks can be useful for the creation of vascularized tissue constructs. In other embodiments, cell-laden hydrogels with embedded sacrificial fiber networks may be produced with the immersed fluidic spinning. The manufactured fiber network can be dissolved and then nutrients can be circulated through the generated fluidic networks until this network is vascularized. Live/dead cell viability studies can be conducted to ensure that the cells will survive in DMEM/EDTA/DSC solution during the time required for calcium alginate fibers dissolution.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” include embodiments that could be described as “consisting essentially of” or “consisting of”, and as such the written description requirement for claiming one or more embodiments of the present invention using the phrase “consisting essentially of” or “consisting of” is met.

What is claimed is:

1) A method of fabricating a microfluidic network, comprising injecting a microfiber material through a needle and into a cross-linking solution, wherein the microfiber material becomes solidified as it is injected into the cross-linking solution.

2) The method of claim 1, wherein the microfiber material is deposited in relatively straight segments or loops to produce a densely coiled stochastic fiber network.

3) The method of claim 2, wherein a diameter of the microfiber material segments or loops ranges from about 10 microns to about 100 microns.

4) The method of claim 2, wherein a diameter of the microfiber material segments or loops ranges from about 100 microns to about 1 mm.

5) The method of claim 1, wherein the microfiber material comprises a carbohydrate alginate or a calcium alginate.

6) The method of claim 1, wherein the needle is immersed in the cross-linking solution.

7) The method of claim 1, wherein the cross-linking solution is a calcium chloride solution.

8) A method of fabricating a tissue construct, comprising:
a. injecting a microfiber material through a needle and into a cross-linking solution, wherein as the microfiber material is ejected from the needle and exposed to the crosslinking solution, it becomes solidified to form a fiber network;
b. removing the fiber network from the crosslinking solution;
c. embedding the fiber network into a cell-laden hydrogel;
d. polymerizing the cell-laden hydrogel; and
e. dissolving said fiber network to leave behind microfluidic channels in the cell-laden hydrogel suitable for vascularization.

9) The method of claim 8, wherein the microfluidic channels are suitable for delivering nutrients to the cells and transporting waste away from the cells.

10) The method of claim 8, wherein the fiber network is dissolved in Ethylenediaminetetraacetic acid (EDTA).

11) The method of claim 8, wherein the microfiber material comprises a carbohydrate alginate or a calcium alginate.

12) The method of claim 8, wherein the needle is immersed in the cross-linking solution.

13) The method of claim 8, wherein the cross-linking solution is a calcium chloride solution.

14) The method of claim 8, wherein the microfiber material is deposited in relatively straight segments or loops to produce a densely coiled stochastic fiber.

15) The method of claim 8, wherein a diameter of the microfiber material segments or loops is smaller than an internal diameter of the needle.

16) The method of claim 8, wherein a diameter of the microfiber material segments or loops ranges from about 10 microns to about 100 microns.

17) The method of claim 8, wherein the microfiber material diameter ranges from about 100 microns to about 1 mm.

18) The method of claim 8, wherein the cell-laden hydrogel is polymerized by photopolymerization.

19) A vascularized tissue construct produced by:
a. injecting a microfiber material through a needle that is immersed in a cross-linking solution, wherein as the microfiber material is ejected from the needle and exposed to the crosslinking solution, it becomes solidified to form a fiber network;
b. removing the fiber network from the crosslinking solution;
c. embedding the fiber network into a cell-laden gel matrix;
d. polymerizing the cell-laden gel matrix; and
e. dissolving said fiber network to leave behind microfluidic channels in the cell-laden gel matrix suitable for vascularization.
